(19) United States
(12) Patent Application Publication
(10) Pub. No.: US 2008/0270188 A1
(43) Pub. Date: Oct. 30, 2008

(54) Graphical display of patient data

(75) Inventors: Chaya Garg, Plymouth, MN (US); William K. Wenger, St. Paul, MN (US); Sara Hoeppner, Burnsville, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/789,690

(22) Filed: Apr. 25, 2007

(51) Int. Cl. A61B 5/00

(52) U.S. Cl. 705/3; 345/418

(57) ABSTRACT

A dynamic graphical display for presenting patient data received from a medical device automatically sorts the patient data into a Venn diagram type format. In one embodiment, the graphical display includes two or more graphic objects representing different types of physiological parameters or events derived from the physiological parameters. The graphic objects define at least a first section corresponding to a first subset of patient data, a second section corresponding to a second subset of patient data, and a third section corresponding to a conjunction of the first and second subsets of patient data. In some embodiments, a user may select one of the sections of the graphical display to obtain further details about the respective subset of patient data or conjunction of subsets of data.

### TECHNICAL FIELD

The invention relates to information visualization, and more particularly, a graphical display of data.

### BACKGROUND

Some medical devices are configured to obtain and store patient data for later retrieval and analysis. The patient data may relate to physiological events such as the onset of bradycardia or tachycardia, the delivery of electrical stimulation (e.g., a defibrillation stimulation) to the patient or physiological parameters, such as electrical activity of the heart, minute ventilation, cardiovascular pressure measures, venous oxygen levels, blood pressure, temperature, and so forth. Other examples of stored information that may generally be referred to as “patient data” include arrhythmia diagnostics, histograms of paced and sensed events, electrograms, such as an electrocardiogram (ECG) or an electroencephalogram (EEG), trends of lead impedance, events marked by the patient (i.e., by providing input via a user interface of a programming device or a medical device). The medical devices may be external, implantable or a combination thereof.

The medical device may store the patient data automatically (e.g., at regular time intervals or upon the occurrence of a particular event) and/or at the direction of the patient or clinician. For example, if the patient senses an onset of a bradycardia or tachycardia event, the patient may activate a data recording feature of a medical device via a patient programmer or another device that is capable of communicating with the medical device. The data recording feature may be activated, for example, by pushing a button on the patient programmer.

The stored patient data may be useful to optimize programming of the medical device, as well as in the management of the patient's arrhythmias and other conditions. A physician may retrieve the patient data from the medical device and analyze the data, for example, to formulate a diagnosis or treatment for the patient. The data may be retrieved from the medical device via an interrogation device that is configured to communicate with the medical device, such as a patient programmer or clinician programmer.

### SUMMARY

In general, the invention is directed toward a graphical display for presenting patient data received from a medical device or manually entered by a clinician, patient or another user. Rather than presenting the patient data in a linear format, the graphical display presents the patient data in a more meaningful format that enables a clinician to more quickly review and ascertain relevant data records, as well as relationships between the different data records. The graphical display includes a plurality of graphic objects arranged in a Venn diagram format, where each graphic object of the Venn diagram corresponds to a type or subset of patient data, and regions of overlap between the graphic objects correspond to a conjunction of the respective data records of each type or subset of patient data. Conditions for sorting patient data into the overlapping sections of the Venn diagram may vary in different embodiments, and in some cases, may be specified by a user. For example, the sections of overlap between graphic objects may correspond to data records for the respective types of physiological parameters or patient events that occurred within a particular time range, such as events that occurred at substantially the same time or within a specified range of time of each other.

In some embodiments, each section (i.e., overlapping and isolated regions of each graphic object) of the graphical display provides a dynamic link, e.g., to a display presenting more detailed information about the patient data records associated with the section. A clinician or user may select a section of the graphical display with a mouse, stylet or another suitable selection technique. Selection of a section of the graphical display may, for example, result in a display including more detailed information about the selected section.

The graphical display may present any suitable patient data, such as raw physiological parameter measurements, events derived from the raw physiological parameter measurements (e.g., physiological parameter measurements exhibiting certain morphological characteristics) or patient markers (e.g., the date and time of patient feedback indicating the occurrence of an event). Raw physiological parameter measurements may be, for example, signals generated by a physiological parameter sensor or signals from the sensor that have been processed to generate quantitative values. Physiological parameter measurements may be, for example, electrocardiogram (ECG) measurements, electroencephalogram (EEG) measurements, electrical activity of the heart, minute ventilation, cardiovascular pressure measures, venous oxygen levels, blood pressure, and body temperature. A patient marker may be, for example, an value, flag, or signal that is stored within a device or transmitted to a device to indicate the occurrence of a certain event, such as the onset of a symptom.

Events may be derived from the physiological parameter measurements, such as physiological parameter values that meet a user-defined condition. In some cases, the event may be more meaningful than the raw physiological parameter measurement. One type of event is defined by a relationship between a physiological measurement and a numerical value. For example, the event may be defined as one or more physiological parameter measurements at, above, or below user-defined thresholds. The amplitude component of a signal indicative of the physiological parameter value may be compared to the threshold.

As other examples, the event may be defined by the relationship between a physiological measurement to which a mathematical operation is applied and some numeral value or as physiological parameter measurements that exhibit a certain characteristic. In some embodiments, a signal indicative of the physiological parameter may be analyzed for slope, amplitude, temporal correlation or frequency correlation with a template signal, or combinations thereof in order to determine whether a certain characteristic is present in the measured physiological parameter. The graphical display may then present the physiological parameter measurements that exhibit the characteristic.

In some embodiments, the dynamic graphical display is presented on a computing device, such as a medical device programmer, that is configured to communicate with an external or implantable medical device. The medical device is configured to record patient data, such as physiological parameter measurements and/or patient markers, either automatically or at the direction of a patient or clinician. The computing device may interrogate the medical device to obtain patient data stored therein. A computer program running on the computing device may then sort through the patient data and present the patient data to a clinician or another user via the dynamic graphical display.

The graphical display described herein presents patient data in a more meaningful way than a linear presentation (e.g., a table, timing diagram or histogram) of the patient data. The graphical display illustrates relationships between different types of patient data, such as the concurrence of two or more events, which allows the clinician to more quickly review and ascertain relevant information.

In one embodiment, the invention is directed to a method comprising receiving patient data from a medical device, generating a graphical user interface comprising plurality of graphic objects, and presenting the graphical user interface on a display. The plurality of graphic objects define at least a first section corresponding to a first subset of patient data, a second section corresponding to a second subset of patient data, and a third section corresponding to a conjunction of the first and second subsets of patient data.

In another embodiment, the invention is directed to a system comprising a display, a user interface, a telemetry module configured to receive patient data from a medical device, a memory configured to store the patient data, and a processor. The processor is configured to generate a graphical user interface comprising plurality of graphic objects defining at least a first section corresponding to a first subset of patient data, a second section corresponding to a second subset of patient data, and a third section corresponding to a conjunction of the first and second subsets of patient data. The processor is further configured to display the graphical user interface on the display.

In another embodiment, the invention is directed to a graphical display including a first graphic object corresponding to a first subset of patient data, a second graphic object corresponding to a second subset of patient data, a third graphic object corresponding to a third subset of patient data, and a fourth graphic object corresponding to a fourth subset of patient data. The first, second, third, and fourth graphical objects partially overlap. A first region of overlap between the first and third graphic objects corresponds to a conjunction of the first and third subsets of data. The first region is represented as a subsection of the first graphical object, and the third graphic object defines a second region corresponding to the first region.

In another embodiment, the invention is directed to a computer-readable medium comprising instructions. The instructions cause a processor to organize patient data received from a medical device into at least a first subset and a second subset of patient data and generate a graphical user interface comprising a plurality of graphic objects defining at least a first section corresponding to the first subset of patient data, a second section corresponding to the second subset of patient data, and a third selectable section corresponding to a conjunction of the first and second subsets of patient data.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram of implantable medical device (IMD) that is implanted in a patient.

FIG. 2 is a conceptual diagram of one type of system in which a clinician remotely receives patient data stored in an IMD.

FIG. 3 is a block diagram illustrating an example configuration of a programmer.

FIG. 4 illustrates a presentation of patient data in a linear format, where each column of the table displays patient data for an event.

FIG. 5 illustrates one embodiment of a dynamic graphical display that presents patient data in a Venn diagram format.

FIG. 6 is another embodiment of dynamic graphical display that includes text indicating the type of patient data represented by each graphic object, as well as a quantitative indicator indicating the number of occurrences within each subset.

FIG. 7 illustrates another embodiment of a graphical display that organizes patient data from four events.

FIG. 8 is a screen shot of an embodiment of a graphical user interface (GUI) presented by software or firmware executed by a processor of programmer or another computing device, where the GUI includes a dynamic graphical display of patient data.

FIG. 9 is a flow diagram of one embodiment of a method for sorting patient data and presenting the patient data in a Venn diagram type graphical display.

### DETAILED DESCRIPTION

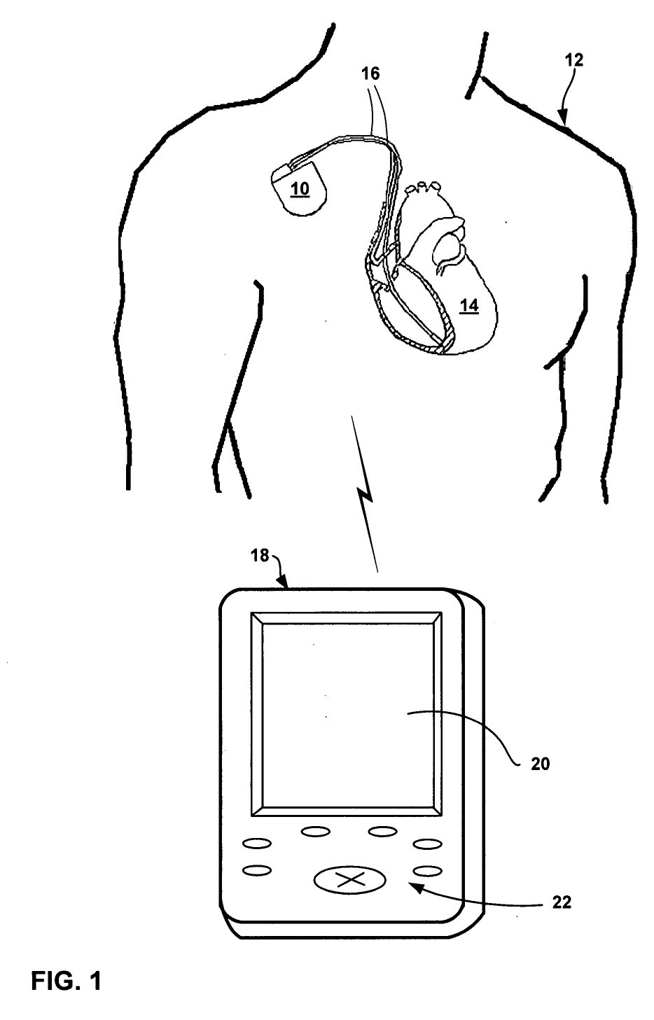

FIG. 1 is a schematic diagram of an implantable medical device (IMD) 10 that is implanted in a patient 12. IMD 10 may be, for example, an electrical stimulator that is configured to provide electrical stimulation to heart 14 of patient 12 via stimulation electrodes carried by medical leads 16, which are electrically and mechanically coupled to IMD 10. For example, IMD 10 may be a pacemaker.

Leads 16 may also carry sensing electrodes in addition to or instead of stimulation electrodes that are configured to sense physiological parameters of patient, such as electrocardiogram (ECG) measurements, electroencephalogram (EEG) measurements, electrical activity of the heart, minute ventilation, cardiovascular pressure measures, venous oxygen levels, blood pressure, body temperature, and so forth. In some cases, electrodes carried by leads 16 may act as both stimulation and sensing electrodes. Furthermore, a housing of IMD 10 may carry stimulation and/or sense electrodes in addition to or instead of electrodes carried by lead 16.

In addition to or instead of physiological parameter sensors on a housing of IMD 10 or carried by leads coupled to IMD, one or more physiological parameter sensors may be in a separate housing from IMD 10 and coupled to IMD 12 via wired or wireless communication. For example, a sensing device may be implanted in another region of patient from IMD 10 or the sensing device may be an external device (e.g., an electrocardiogram (ECG) belt, a respiration belt, a motion sensor such as an accelerometer (e.g., to indicate a patient activity level or posture) or a transducer that outputs a signal as a function of the oxygen saturation of the blood of patient 12).

IMD 10 includes volatile or non-volatile memory, such as a random access memory (RAM), read-only memory (ROM), non-volatile random access memory (NVRAM), electrically erasable programmable read-only memory (EEPROM), flash memory, and the like to store stimulation parameters for IMD 10 as well as patient data obtained by IMD 10. IMD 10 may obtain and store different types of patient data from patient 12 over a period of time, such as between clinic visits. The patient data may include any data that may be meaningful to a clinician for rendering a diagnosis, for evaluating a therapy delivered by IMD 10 or other data relating to the physiological condition or symptoms of patient 12.

The patient data may include, for example, physiological parameter measurements obtained via the sensing electrodes carried by leads 16 or the housing of IMD 10, information about the occurrence of the event, such as the time the event occurred, and/or diagnostic data about IMD 10, such as the remaining battery life. The physiological parameter measurements may be automatically recorded on a regular basis or upon the occurrence of a triggering event, such as when defibrillation stimulation is delivered to heart 14, or at the direction of patient 12 or a clinician. Patient data recorded by IMD 10 may also include patient markers, which may include, for example, a value, flag, or signal that generated upon receiving feedback from patient 12 to indicate the occurrence of a certain event, such as the onset of a symptom. The data recorded by IMD 10, whether physiological parameter measurements, the time an event occurred, patient markers or otherwise, is generally referred to as “patient data” throughout the disclosure.

The patient data may also include events that are derived from the physiological parameter measurements, such as physiological parameter values that meet a user-defined condition. In some cases, the event may be more meaningful than the raw physiological parameter measurement. Examples of different types of patient events are described in further detail below.

IMD 10 is just one example of a medical device that records patient data. Other examples of IMDs include electrical stimulators (e.g., neurostimulators), fluid delivery devices, such as drug pumps, and sensing devices. The patient data organization technique and dynamic graphical display described herein are applicable to any patient data, regardless of the origin. Thus, the present invention is also applicable to patient data obtained automatically or manually from external medical devices. As previously described, patient data received from an external medical device may include, for example, sensor signals from an external activity sensor (e.g., an accelerometer) that indicates a patient activity level, an external physiological parameter sensor (e.g., a respiration belt, a transducer that outputs a signal as a function of the oxygen saturation of the blood of the patient or an ECG belt that incorporates a plurality of electrodes for externally sensing the electrical activity of the heart of the patient).

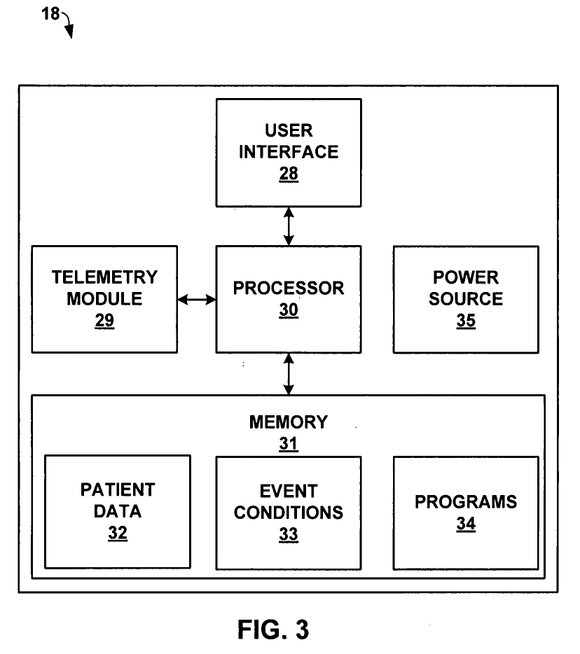

A clinician or patient 12 may interrogate IMD 10 with programmer 18 in order to retrieve patient data stored within IMD 10. Programmer 18 may be, for example, a patient programmer, a clinician programmer or any other computing device configured to communicate with IMD 10 via wired or wireless telemetry. In the embodiment shown in FIG. 1, programmer 18 is configured to interrogate IMD 10 and download the data stored within the memory of IMD 10 via wireless telemetry techniques, such as by radio frequency (RF) communication. As described in further detail with reference to FIG. 3, the downloaded patient data may be stored in memory of programmer 18. The patient data may also be displayed on display 20 of programmer 18, and a clinician or patient 12 may scroll through and manipulate the patient data using user interface 22, which may include buttons, a keyboard, a touchpad or another user interface. The clinician may analyze the downloaded patient data when formulating a diagnosis or, treatment plan for patient 12 or to determine the status of IMD 10.

The clinician may access the patient data stored within IMD 10 via programmer 18 when the clinician is in close proximity to patient 12. Alternatively, the clinician may remotely retrieve patient data from IMD 10. Remote interrogation of IMD 10 may allow the clinician to monitor the condition of patient 12 or IMD 10 without requiring patient 12 to be physically present in the clinician's office.

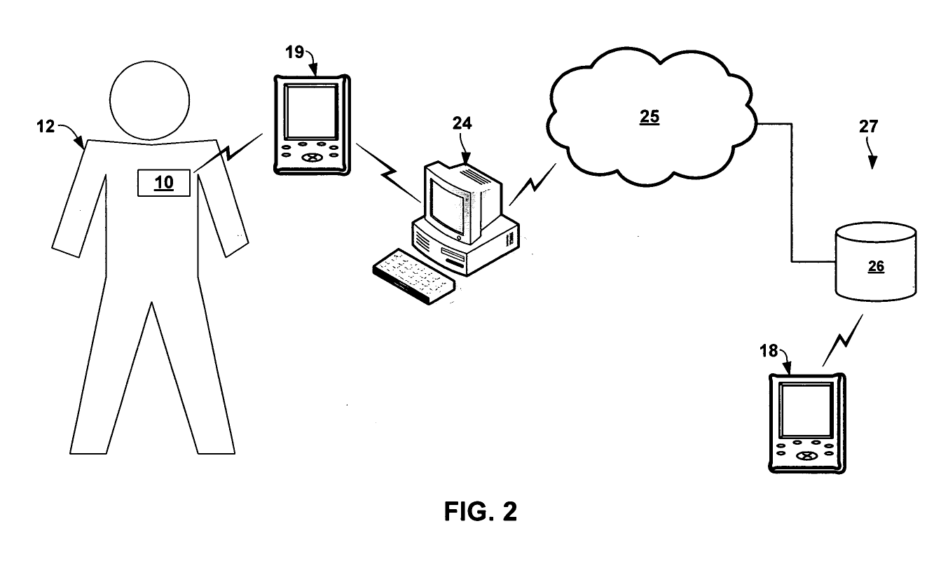

FIG. 2 is a conceptual diagram of one type of system in which a clinician remotely receives patient data stored in IMD 10. After retrieving data from IMD 10, a patient programmer 19 may upload the data to a computing device 24, which may be, for example, a personal computer that is connected to communications network 25, such as a public switched telephone network (PSTN), a cellular communication network, a radio communication network, the Internet, or some other communication network. Computing device 24 transmits the patient data through network 25 to a clinician database 26. Alternatively, patient programmer 19 may directly transmit the information through network 25 to clinician database 26. At the clinician end 27 of network 25, the patient data may not directly be stored into clinician database 26, but rather the patient data may be routed through servers, computing devices, and so forth, depending on the particular type and arrangement of clinician database 26. The clinician may transfer the patient data from clinician database 26 onto programmer 18 in order to view and analyze the patient data. Alternatively, the clinician may view and analyze the patient data via another type of computing device, such as a personal computer.

FIG. 3 is a block diagram illustrating an example configuration of programmer 18, which includes user interface 28, telemetry module 29, processor 30, memory 31, and power source 35. Telemetry module 29 permits communication with IMD 10 for transfer of data and adjustment of any therapy delivery parameters. The therapy delivery parameters may be stimulation parameters in the case of IMD 10 or fluid delivery parameters in the case of a fluid delivery device, such as a drug pump. Telemetry module 29 may include a transmitter and receiver to permit bi-directional communication with IMD 10. Memory 31 stores patient data 32 as well as event condition definitions 33 and program instructions 34 that, when executed by processor 30, cause programmer 18 to perform functions such as displaying patient data 32 via a dynamic graphical display. In some embodiments, such as in the case of the remote patient data retrieval system shown in FIG. 2, the clinician may execute the program instructions on a computing device in order to review patient data stored in clinician database 26. In addition, therapy programs created by the clinician may be stored in programs section 34 of memory 31. Memory 31 may include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like. Power source 35 may take the form of a rechargeable or non-rechargeable battery.

A clinician or other user may interact with processor 30 via user interface 28 in order to review and manipulate patient data received from IMD 10. User interface 28 may include display 20 and keypad 22 (FIG. 1), and may also include a touch screen or peripheral pointing devices, such as a mouse. Processor 30 provides a dynamic graphical user interface (GUI) via user interface 28 to facilitate interaction between a clinician and patient data 32 stored within memory 31. Processor 30 may include a microprocessor, a controller, a digital signal processor (DSP), an application specific integrated circuit (ASIC), a field programmable gate array (FPGA), discrete logic circuitry, or the like.

Processor 30 may also transmit programs 34 created by the clinician and stored within memory 31 to IMD 10 via telemetry module 29. In addition, processor 30 may process patient data according to event condition 33 definitions stored within memory 31 of programmer 18 prior to presenting the patient data via a dynamic graphical display. The event definitions may be created and modified by the clinician or another user. The event definitions may be specific to a particular patient, or alternatively, the event definitions may be applied to patient data for more than one patient.

The physiological parameter values or other patient data recorded by IMD 10 may be useful to a clinician, but in some cases, the clinician may be interested in reviewing “events” occurring within the patient data. Events may be derived from the raw physiological parameter measurements, such as physiological parameter values that meet a user-defined condition. One type of event is defined by a relationship between a physiological measurement and a numerical value. For example, the event may be defined as one or more physiological parameter measurements at, above, or below user-defined thresholds. The amplitude component of a signal indicative of the physiological parameter value may be compared to the threshold.

As another example, the event may be defined by the relationship between a physiological measurement to which a mathematical operation is applied and some numeral value. As another example, the event may be defined as physiological parameter measurements that exhibit a certain characteristic. In some embodiments, processor 30 may analyze a waveform of the signal received from the sensing electrodes carried by lead 16 (FIG. 1) to determine whether an event occurred. Particular characteristics of the waveform may be compared to a template in order to determine whether a certain characteristic is present in the measured physiological parameter, and thus, whether an event occurred for the purposes of displaying patient data on a graphical display. As various examples, a trend in the physiological parameter measurement over time may be compared to a template. For example, the physiological parameter may be a blood pressure, and the template may indicate an abnormal trend in blood pressure that precedes a cardiac episode that requires treatment. The template may be predetermined by a user and stored within a memory of IMD 10 or memory 31 of programmer 18.

As examples of different techniques for comparing a trend to a template, a signal from one or more sensors may be analyzed for slope. If the time rate of change (i.e., the slope) of the physiological parameter measurements over a period of time matches or exceeds the time rate of change of the template, processor 30 may mark an event that is to be included in the graphical display. The period of time may be predetermined by a user, and may be any suitable time span in which the time rate of change may be determined. Alternatively, a correlation between inflection points in the amplitude waveform of the physiological parameter signal or other critical points and a template may indicate an occurrence of an event.

Patient events may also be derived based on a temporal correlation or frequency correlation of a waveform of a physiological parameter signal received from IMD 10 with a template waveform. For temporal correlation, the physiological parameter signal may be sampled within a sliding window and compared to a template to identify a signal that correlates well with the template. For example, processor 30 of the implanted medical device or an external device may perform a correlation analysis by moving a window along a digitized plot of the amplitude of the measured physiological parameter at regular intervals, such as between about one millisecond to about ten millisecond intervals, to define a sample of the physiological parameter signal. The sample window is slid along the plot until a correlation is detected between the waveform of the template and the waveform of the sample of the physiological parameter signal defined by the window. By moving the window at regular time intervals, multiple sample periods are defined. The correlation may be detected by, for example, matching multiple points between the template waveform and the waveform of the plot of the physiological parameter signal over time, or by applying any suitable mathematical correlation algorithm between the sample in the sampling window and a corresponding set of samples stored in the template waveform. If the physiological parameter signal waveform substantially correlates to the template waveform, processor 30 may indicate an occurrence of an event for later presentation to a clinician.

For frequency correlation, the physiological parameter signal may be analyzed in the frequency domain to compare selected frequency components of the physiological parameter signal to corresponding frequency components of the template signal. For example, one or more specific frequency bands may be more revealing than others, and the correlation analysis may include a spectral analysis of the physiological parameter signal component in the revealing frequency bands. The frequency component of the physiological parameter signal may be compared to a frequency component of a template. If the frequency components of the physiological parameter signal waveform substantially correlates to the template waveform, processor 30 may indicate an occurrence of an event for later presentation to a clinician.

In each of the embodiments described above, the one or more templates may be stored within memory 31 of an external device, such as programmer 18 or within a memory of IMD 10. In some embodiments, different templates are employed and selected based on the patient physiological condition, which may be affected by, for example, the time of day or activity (e.g., sleeping, exercising, etc.). Furthermore, in other embodiments, a processor within IMD 10 may derive patient events from the physiological parameter measurements and transmit the event data to programmer 18.

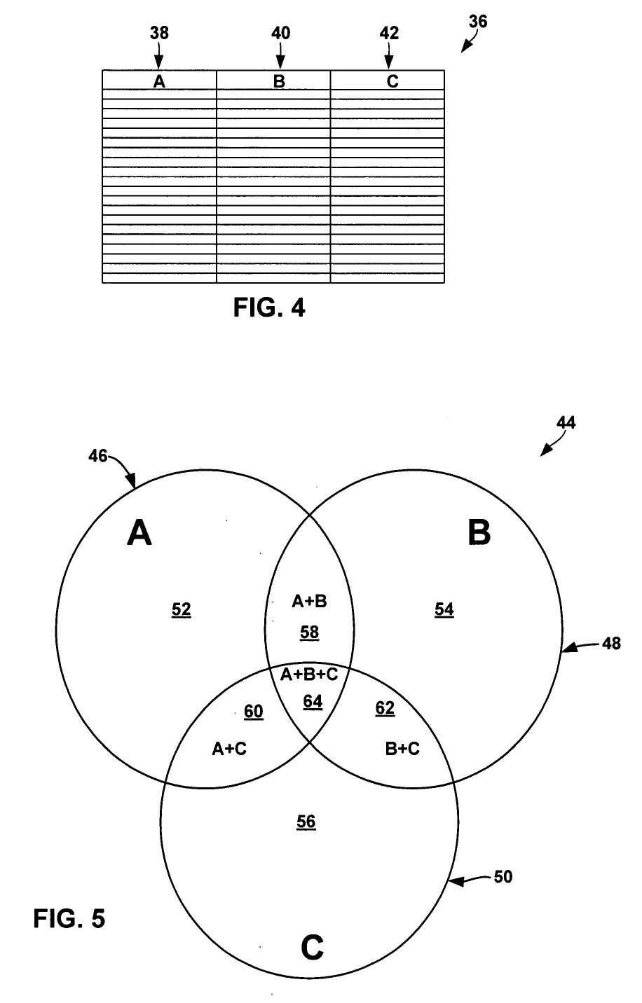

While many existing medical device programmers are configured to retrieve information from IMD 10, the existing programmers typically do not have the ability to automatically track, recognize, and analyze trends in the data downloaded from IMD 10. For example, the existing programmers may display patient data for events A, B, and C in tabular form, as shown in FIG. 4. FIG. 4 illustrates table 36, which has column 38 displaying data for event A, column 40 displaying data for event B, and column 42 displaying data for event C. As an example, each row of each of the columns may display a time at which the event represented by the respective column occurred. The patient data may be listed chronologically (i.e., arranged by the time in which the data was recorded by IMD 10). Patient data may also be provided in other linear formats, such as timing diagrams or histograms.

When the clinician is presented with patient data in tabular form or another linear format, the clinician must typically manually sort through the data in order to analyze the patient data. Oftentimes, a combination of events derived from the patient data may be more meaningful than discrete events. Organizing the patient data presented in a tabular or other linear format into the meaningful combinations of events may be cumbersome. For example, if the clinician is interested in reviewing instances in which the patient's ECG measurement was above a respective threshold value at the same time (or within a certain span of time) the patient's EEG data was above a respective threshold value, the clinician may manually scroll through the linear list and manually sort the patient data that meets those conditions. As another examples, a clinician may be interested in reviewing events in which the morphological features of two physiological parameter signals, such as EEG and ECG signals, indicates a concurrence of events, such as concurrent seizure and arrhythmia. Again, the clinician may manually scroll through a linear list of the physiological parameter data and analyze the data to see whether the seizure and arrhythmia event occurred, and then further associate a seizure event with a concurrent arrhythmia event, or vice versa.

Given the large volume of patient data that may be retrieved from IMD 10 at once, the sorting and analysis process may be time consuming and may entail some degree of human error. In addition, the clinician may not easily be able to identify subtle or even significant trends in data, or identify any relationships between occurrences of different types of data. Rather, such trends may only be apparent after a substantial investment of time into sorting through the patient data and, in some cases, translating the patient data into events of interest.

Programmer 18, however, provides a graphical display that automatically sorts patient data retrieved from IMD 10, enabling the clinician to more quickly review and ascertain relevant data and relationships between the data records. The patient data is typically comprised of a plurality of data records, which may be, for example, an occurrence of an event or a patient marker. FIG. 5 illustrates one embodiment of dynamic graphical display 44 that may be provided by programmer 18 via software or firmware. Graphical display 44 provides a useful interface for accessing patient data 32 (FIG. 3) that may be stored within memory 31 (FIG. 3) of programmer 18.

Dynamic graphical display 44 may present raw patient data and/or events that are derived from the raw patient data, as described in detail above. That is, graphical display 44 may organize the patient data based on the type of data (e.g., ECG data, EEG date, blood pressure data, patient marker data, and so forth). In addition to, or instead of organizing patient data based on the type of data, dynamic graphical display 44 may organize the patient data into “events” of interest to the clinician. Events are generally measures that are derived from the raw patient data, such as physiological parameter measurement that meet a user-defined condition or the occurrence of a signal indicative of the physiological parameter measurement that exhibits a certain characteristic.

One type of event is defined by a relationship between a physiological measurement and a numerical value. For example, the event may be defined as one or more physiological measurements having a frequency or amplitude at, above, or below user-defined threshold values. As another example, an event may be defined by the relationship between a physiological measurement to which a mathematical operation is applied and some numeral value. For example, in one embodiment, the event may be defined as an average value of one physiological parameter measured over a predetermined time span, such as five to ten minutes. Another type of event is defined as the occurrence of two or more physiological parameters that have a certain relationship, such as two or more sub-events that meet a particular temporal condition. In one embodiment, the temporal condition is satisfied when an occurrence of a physiological parameter value from a first subset of patient data (e.g., one type of physiological parameter) occurs within a predetermined time range of an occurrence of a physiological parameter value from a second subset of patient data. The predetermined time range may be defined by a user, and may be, for example, less than one minute (e.g., occurring substantially simultaneously) or within fifteen minutes, thirty minutes, an hour, two hours, and so forth. In another embodiment, the temporal condition is satisfied when an occurrence of a physiological parameter value from a first subset of patient data and an occurrence of a physiological parameter value from a second subset of patient data occur in a particular order. Alternatively, as described above, an event may be defined by a relationship between one or more physiological parameter values.

Graphical display 44 includes a plurality of graphic objects 46, 48, and 50 that define a dynamic Venn diagram, where each graphic object 46, 48, and 50 corresponds to sets of data for events A, B, and C, respectively. In other embodiments, one or more sets of data represented by objects 46, 48, and/or 50 may correspond to different types of physiological parameters. In some embodiments, graphic objects 46, 48, 50 may each include a property similar to that of an icon on many common graphical user interfaces for personal computers. In those embodiments, graphic objects 46, 48, 50 are selectable discrete pictograms or symbols or each define more than one selectable discrete pictogram or symbol, which a user make select to navigate to another display, as well as other operations (e.g., moving or “dragging” the icon to a new location within the display).

In order to visually distinguish between each graphic object 46, 48, and 50, each object may have a different visual attribute, such as a different color, line quality or fill pattern. The relative positions between of objects 46, 48, and 50 visually convey the relationship between the sets of data represented by each object 46, 48, and 50. In particular, for the three events A, B, and C, graphical display 44 organizes the set of patient data into subsets of data relating to event A, event B, event C, and overlaps in events A, B, and C. Each subset of patient data is schematically represented by sections defined by the intersection (or conjunction) of the respective objects 46, 48, and 50 and the remaining sections of objects 46, 48, and 50 that do not overlap. That is, each of the areas within objects 46, 48, and 50 that do not overlap with another object corresponds to a subset of patient data for an event A, B or C that is exclusive from the other events A, B or C. Section 52 corresponds to, e.g., times at which only event A occurred, section 54 corresponds to patient data in which only event B occurred, and section 56 corresponds to patient data in which only event C occurred.

In addition to an initial set of conditions that may be used to sort patient data into events A, B, and C, graphical display 44 may further sort the events by their relationship to each other. Accordingly, each of the sections defined by overlapping regions of graphic objects 46, 48, and 50 correspond to subsets of data that include data for at least two events A, B or C, where the data from each of the events A, B or C overlaps in some way with data from another event A, B or C. Conditions for sorting patient data into the “overlapping” sections of graphical display 44 may vary in different embodiments, and in some cases, may be specified by a user. For example, sections defined by overlapping regions of graphic objects 46, 48, and 50 may correspond to events A, B or C that occurred within a predetermined time range (e.g., at substantially the same time or within a specified amount of time of each other).

In one embodiment, section 58 (which represents the overlap between graphic objects 46 and 48) corresponds to patient data in which events A and B occurred at substantially the same time, during a user-specified amount of time or which meets another user-specified limitation. For example, the user (e.g., the clinician) may specify that section 58 includes patient data in which events A and B occurred with 30 minutes of each other, regardless of which event occurred first. Alternatively, the user may specify that section 58 include patient data in which event A occurred before event B or vice versa. Other limitations on the data within section 58 may also be specified.

Section 60 of graphical display 44 corresponds to patient data in which events A and C occurred within a predetermined time range, which may or may not be the time range for the data within section 58. Section 62 corresponds to patient data in which events B and C occurred within a predetermined time range. Finally, section 64 corresponds to patient data in which events A, B, and C occurred within a predetermined time range. The predetermined time range may be the same as or different for each section 58, 60, 62, and 64.

Each section 52-64 of graphical display 44 is dynamic, and a clinician or another user may select a section to obtain more information about the subset of patient data represented by the section. A clinician may select each section 52-64 with a mouse, stylet or another peripheral device. For example, selecting section 58 may cause patient data in which both events A and B occurred at the same time to be provided in a new display or within the same display. After a user inquires for more detailed information about the events falling within a selected section 52-64, the outputted data may be in any suitable form, such as a table or another linear form. Selectable sections 52-64 of graphical display 44 that are associated with a particular event or relationship between events enable a clinician to query relationships between events A, B, and C without having to enter logical Boolean operators (e.g., AND or OR operators).

If events A, B, and C do not overlap in time or meet another user-specified limitation, the relevant sections 46, 48, and 50 may not overlap and dynamic graphical display 44 may not include at least one section 58, 60, 62, and 64. For example, if events A and C do not overlap, section C may be offset from section A while still overlapping with section B. Alternatively, section 60 may be a “dead” link, and may be provide a nonselectable area that is unavailable for selection by a user. As yet another alternative, section 60 may be an active link, but upon selection of section 60, no information may be presented.

In contrast to table 36 of FIG. 4 or another linear format, dynamic graphical display 44 provides concurrent graphical visualization of patient data relating to events A, B, and C. The organization of patient data into sections that relate the data about one event to other events provides a graphical display 44 that a clinician can view and track the data, quickly ascertain trends in data, and determine relationships between different events without having to invest a substantial amount of time into organizing the patient data. When data is provided in a linear format (e.g., table 36), the clinician may not be able to ascertain details about the patient data as quickly as viewing graphical display 44.

In addition to providing visual indications of relationships between the subsets of patient data for events A, B, and C, graphical display 44 may also be useful for deriving a new indicator of a physiological condition, such as the onset of a bradycardia event, or associating a symptom experienced by patient 12 with a physiological condition. In one embodiment, IMD 10 may record patient data upon the activation of the recording feature by patient 12. If patient 12 feels a symptom, such as pain, faintness or light-headedness, for example, patient 12 may activate the recording feature of IMD 10 by, for example, depressing a button on a programmer 18 or 19, or another communication device. In response to the recording feature activation, IMD 10 may record one or more physiological parameter values occurring at that time. The clinician may determine which physiological parameters are recorded in response to the activation of the recording feature by patient 12. In this way, IMD 10 records relevant data, and the clinician may later associate the data recorded by IMD 10 with a patient symptom. The clinician may use the recorded information to determine whether the patient 12 actually experienced a physiological condition and requires immediate attention.

In another embodiment, IMD 10 may merely mark the time and date that the patient 12 felt the symptom. If patient 12 feels a symptom, patient 12 may depress a button on programmer 18 or 19, or another communication device, and IMD 10 may record the “patient marker,” such as by storing a value, flag, or signal. Alternatively, programmer 18 may record the patient marker. Patient marker information may help the clinician later diagnose the symptom of patient 12 at the time the patient marker was created. The clinician may associate the patient marker with other events in order to see whether patient 12 exhibited a physiological condition that would account for the symptom felt by patient 12. Graphical display 44 may be useful for associating the patient marker with other events without requiring the clinician to invest a large amount of time into sorting the patient data.

For example, graphical display 44 may organize the patient data into event A, which may be the time and date of patient markers, event B, which may be ECG events (e.g., ECG measurements at, below, or above a threshold), and event C, which may be EEG events (e.g., EEG measurements at, below or above a threshold). The region of overlap (i.e., section 58) between object 46 (i.e., event A) and object 48 (i.e., event B) may provide an active link to ECG data that IMD 10 recorded at or around the same time as the patient marker. Similarly, section 60 between object 46 and object 50 may provide an active link to EEG data that IMD 10 recorded at the time of the patient marker. Section 62, which is the region of overlap between objects 48 (event B) and 50 (event C) may provide an active link to EEG events and ECG events recorded at substantially the same time (or within a certain amount of time of each other), but the EEG and ECG events were not accompanied by a patient marker. Finally, section 64, which is the region of overlap between all three objects 46, 48, and 50, may provide an active link to ECG data and EEG data that IMD 1 0 recorded at the time of the patient marker.

Some clinicians utilize a remote patient data acquisition system, such as the schematic system shown in FIG. 2, in order to remotely monitor more than one patient. For example, patient data may be gathered for multiple patients over a period of time, such as over one or more days, and the clinician may review the patient data in order to assess the state of the patient's health. The remote retrieval of patient data from each patient's medical device may be an efficient system for monitoring multiple patients because the clinician may review the patient data for multiple patients without having to examine or interview each patient individually. A clinician may sort patient data for multiple patients via graphical display 44 to determine which, if any, patients require priority of attention over other patients.

In FIG. 5, sections 46, 48, and 50 of the Venn diagram may each represent a physiological parameter event or a patient marker event A, B, and C, respectively, while the data within each selectable area 52-64 includes patient names and/or other identifying information, such as contact information for the respective patient. The clinician may be able to quickly ascertain which patients exhibited only one event A, B or C, two events A, B, and/or C, or all three events A, B, and C during the time period represented by the patient data retrieved by the clinician. Depending on the particular symptoms or events represented by graphic objects 46, 48, and 50, the occurrence of a particular combination of symptoms or events may represent the most problematic patient condition, and the clinician may choose to focus attention on the patients exhibiting the particular combination of symptoms or events. For example, if event A represents pain (i.e., the patient created a patient marker via a programmer 18 or 19 upon feeling pain), event B represents the occurrence of delivery of defibrillation stimulation to the heart, and event C represents a sensed physiological parameter value above a certain threshold, the clinician may determine that patients within section 64 require attention before any of the other patients. Section 64 represents in the intersection of sections 46, 48, and 50 and patients for which events A, B, and C occurred.

In order to visually distinguish between each section 52, 54, 56, 58, 60, 62, and 64, two or more of the sections may have a different visual attribute, such as a different color, line quality or fill pattern. In addition, to further aid a user in identifying relationships between the sets of data represented by graphic icons 46, 48, and 50, in some embodiments, sections 58, 60, 62, and 64 that represent regions of “overlap” between graphic icons 46, 48, and 50 may have different sizes. In one embodiment, the relative size between the sections 58, 60, 62, and 64 may indicate the relative number of events that are represented by each section 58, 60, 62, and 64. As one example, if section 58, which represents the overlap between events A and B, includes twice as many events/data records as section 62, which represents the overlap between events B and C, section 58 may be twice as large as section 62. Non-proportional sizing (e.g., in the previous example, section 58 may not be twice as large as section 62, but merely large enough to show the relative difference in event numbers) may also be used, so long as a user may easily identify the relative difference in quantity of events associated with each section.

Furthermore, color-coding or another coding technique may be used in addition or instead of the relative sizing. For example, a first color may indicate that a section includes 0-10 data records, while a second color may indicate that a section includes 11-20 data records, and so forth. The number of data records represented by each color may be selected by a user or by a manufacturer of graphical display 44, and may be pre-set or modifiable as the needs of the user change.

In other embodiments, a size of each section 52, 54, 56, 58, 60, 62, and 64, rather than just sections 58, 60, 62, and 64, may depend upon the number of events associated with each section 52, 54, 56, 58, 60, 62, and 64. If events A, B, and C each include a different quantity of data records, objects 46, 48, and 50 may also be sized relative to each other or color-coded to indicate the number of data records of events A, B, and C, respectively.

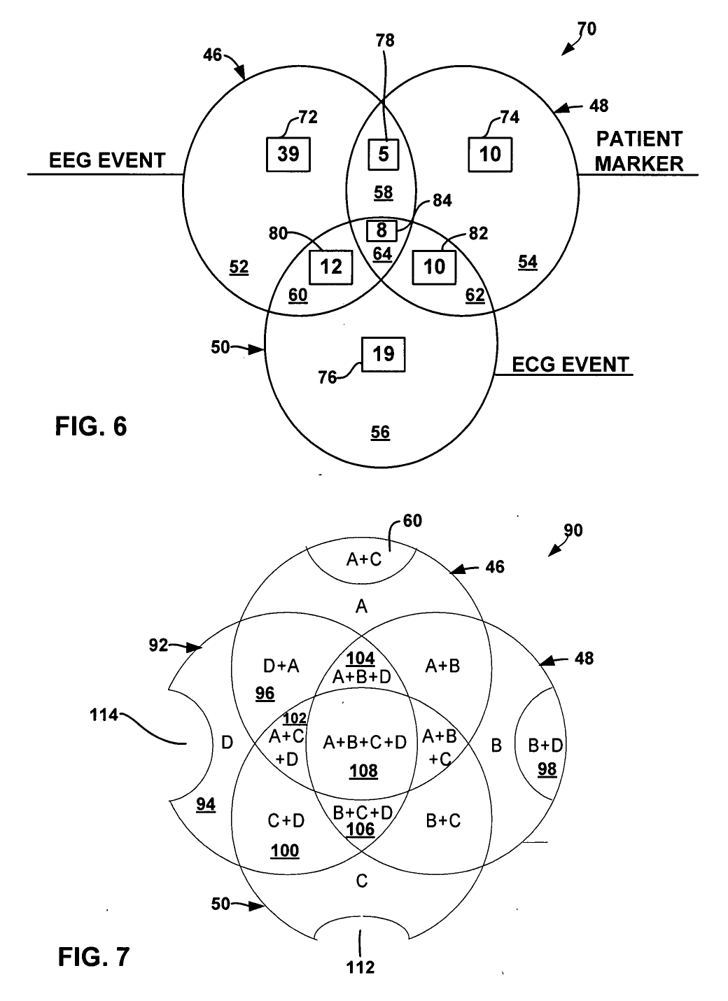

FIG. 6 is another embodiment of dynamic graphical display 70, which is similar to graphical display 44 of FIG. 5, except that graphical display 70 includes text that indicates the type of patient data within each object 46, 48, and 50. In addition, graphical display 70 includes quantitative indicators indicating the number of occurrences of each event A, B, and C within each section 52-64. In the embodiment shown in FIG. 6, graphic object 46 represents patient data relating to an EEG event, such as an EEG measurement above a predetermined threshold or a seizure, and graphic object 50 represents patient data relating to an ECG event, such as an ECG measurement above a predetermined threshold, a tachycardia or other arrhythmia. Graphic object 48 represents patient data recorded upon the activation of the recording feature on a programmer 18 or 19 (FIGS. 1 and 2) by the patient. For example, when patient 12 feels chest pain, patient 12 may depress a button or otherwise interact with a programmer, which then instructs IMD 10 (FIG. 1) to record the date and time of the patient marker, or a sensed physiological parameter value.

Onsets of various physiological conditions are typically associated with patient symptoms. As one example, onset of bradycardia, an abnormally slow or irregular heartbeat, may be associated with symptoms, such as fatigue, dizziness or fainting. If patient 12 feels a symptom and indicates so via a programmer 18 or 19, IMD 10 may record physiological data occurring at the time that patient 12 feels the symptoms, or alternatively, IMD 10 may merely record the date and time at which patient 12 felt the symptoms. Recording the time and date may help a clinician later identify what type of physiological condition caused patient 12 to feel the symptoms. In addition, the clinician may associate the time and date of the patient symptom with measured physiological parameters. Recognizing a relationship between the measured physiological parameter values and the patient symptoms may help the clinician formulate a diagnosis and a therapy program for patient 12.

Graphical display 70 includes text describing the type of data that is within each object 46, 48, and 50, which may help a clinician visually identify the data within each object 46, 48, and 50. In addition, graphical display 70 includes indicators 72, 74, 76, 78, 80, 82, and 84 that display the number of occurrences that are within the respective section 52-64. As with the textual descriptions of each graphic object 46, 48, and 50, indicators 72-84 enable a clinician to easily identify the number of overlaps between the subsets of data represented by each graphic object 46, 48, and 50. For example, in the example graphical display 70 shown in FIG. 6, the clinician may, without having to manually manipulate data, determine that an ECG measurement above a respective threshold occurred 19 times without the occurrence of an EEG measurement above a respective threshold, or without a patient marker. The clinician may also determine that events A, B, and C occurred at substantially the same time, within a certain amount of time or in a particular order eight times. A user may select any section 52-64 or indicator 72-84 to review actual data values of the respective section.

A dynamic graphical display in accordance with the invention may organize patient data relating to any suitable number of events. FIG. 7 illustrates another embodiment of a graphical display 90 that organizes patient data from four events A, B, C, and D. In addition to graphic objects 46, 48, and 50 from graphical displays 44 and 70 of FIGS. 5 and 6, respectively, graphical display includes graphic object 92, which represents data from event D. Together, objects 46, 48, 50, and 92 overlap to define selectable sections 52-64 (labeled in FIGS. 4 and 5), as well as sections 94, 96, 98, 100, 102, 104, 106, and 108.

Section 94 of graphic object 92 corresponds to patient data in which event D occurred without overlapping in time or meeting another user-specified limitation with the other events A, B, and C. Section 96 provides a dynamic link to data in which events A and D occurred at substantially the same time or data meeting another user-specified limitation. Section 98 provides a dynamic link to data in which events B and D occurred at substantially the same time or data meeting another user-specified limitation. Section 100 provides a dynamic link to data in which events C and D occurred at substantially the same time or data meeting another user-specified limitation. Section 102 provides a dynamic link to data in which events A, C, and D occurred at substantially the same time or data meeting another user-specified limitation. Section 104 provides a dynamic link to data in which events A, B, and D occurred at substantially the same time or data meeting another user-specified limitation. Section 106 provides a dynamic link to data in which events B, C, and D occurred at substantially the same time or data meeting another user-specified limitation. Section 108 provides a dynamic link to data in which events A, B, C, and D occurred at substantially the same time or data meeting another user-specified limitation.

Increasing the number of events displayed by dynamic graphical display 90 increases the complexity of graphical display 90. In particular, the greater the number of patient data events, the greater the number of overlapping objects (i.e., more regions of overlap). In order to help a user more easily identify each section 52-64 and 94-110, graphical display 90 is arranged such that section 60, representing the region of overlap between events A and C (i.e., between graphic objects 46 and 50) and section 98, representing the region of overlap between events A and D (i.e., between graphic objects 46 and 92) are separated from the remaining sections 52-56, 62, 64, 94, 96, and 100-108.

A cutout 112 in object 50 that corresponds to the overlapping section 60 in graphic object 46 may help visually direct the user to section 60, which opposes cutout 112. In one embodiment, cutout 112 is represented as a void in object 50, while overlapping section 60 is presented as a subsection of graphic object 46. In other embodiments, cutout 112 may be presented to a user using any other technique that distinguishes cutout 112 from graphic object 50. For example, in other embodiments, cutout 112 may be a subsection of graphic object 50 or include coloring, shading or other characteristics that distinguish cutout 112 from graphic object 50. In embodiments in which sections of display 90 are selectable, the user may select section 60 to receive further information about the conjunction of events A and C. Cutout 112 may or may not be selectable in some embodiments. For example, in one embodiment, if the user selects cutout 112 to inquire about the conjunction of data records of events A and C, the user may be automatically directed to section 60.

Similarly, cutout 114 in object 92, which corresponds to section 98, may help visually direct the user to section 98, which opposes cutout 114. Section 98 represents a section (or region) of overlap between events B and D. Accordingly, cutout 114 in object 92 corresponds to the region of overlap between events B and D, i.e., section 98, and section 98 is represented as a subsection of object 48. In embodiments in which each section of display 90 is selectable, the user may select section 98 to receive further information about the conjunction of events B and D. The graphical display including a first region that corresponds to a conjunction of two data sets (e.g., section 98) and a second region that corresponds to the first region (e.g., section 114) may be used in graphical displays of any types of data, and is not limited to medical data.

While dynamic graphical displays organizing patient data from three or four events is shown in FIGS. 5-7, in other embodiments, a graphical display may organize patient data from any suitable number of events. Examples of Venn diagrams that may be used to represent five or more sets of data (e.g., events) are Edwards-Venn diagrams.

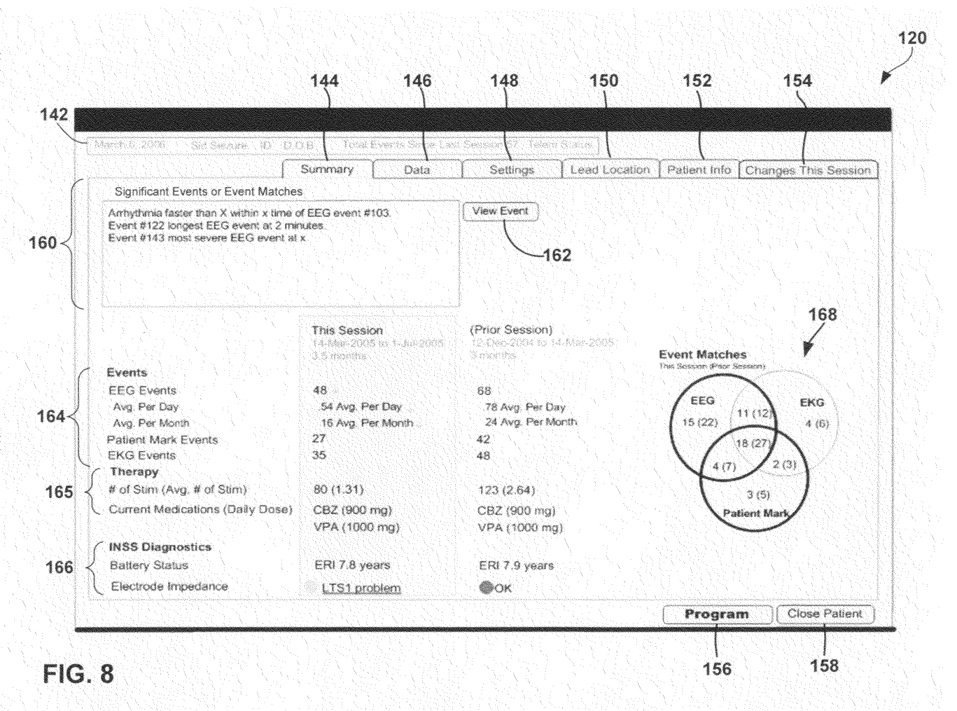

FIG. 8 is a screen shot of an embodiment of a graphical user interface (GUI) 140 presented by software or firmware executed by a processor of programmer 18 for IMD 10 (FIG. 1) or another computing device. GUI 140 incorporates dynamic graphic display 168 similar to graphical displays 44 and 70 of FIGS. 5 and 6, respectively. Upon interrogating IMD 10 and retrieving patient stored within IMD 10, programmer 18 may present the patient data via GUI 140. GUI 140 may also be periodically updated in real-time as new patient data is received from IMD 10.

GUI 140 comprises information bar 142, which may display relevant textual information, such as the current date, the name of the patient, date of birth, and the total number of events since the last telemetry session between programmer 18 and IMD 10. In addition, GUI 140 includes display pages 144, 146, 148, 150, 152, and 154, and selection buttons 156 and 158. The clinician may select selection button 156 in order to change the therapy program for patient 12, which may include, for example, adjusting the stimulation parameters of IMD 10. The clinician may select button 158 in order to end the session for patient 12. The “session” may refer to the session for reviewing the patient data retrieved from IMD 10, or to the time period in which programmer 18 is in communication with IMD 10 (e.g., a programming session).

Summary page 144 provides a summary of the patient data received from IMD 10, while display page 146 provides the patient data in a linear format (e.g., table 36 in FIG. 4) or in an organized dynamic display, such as the Venn diagrams type graphical displays shown in FIGS. 5-8C above. Summary page 144 generally provides a summary of the patient data retrieved from IMD 10, where the patient data is organized into a format that a clinician may review and quickly ascertain the relationship between different events recorded by IMD 10. In the embodiment shown in FIG. 8, summary page 144 includes a listing of significant events or event matches 160, selection button 162, a listing of events 164, a listing of therapy 165 provided by IMD 10 for the current session and the prior session, and a summary of the data relating to the events for the current session and a previous session, a diagnostic summary 166 of IMD 10 for the current session and a previous session, and a dynamic graphical display 168 of the patient data.

A clinician may an event or event matching within the significant events or event matches listing 160 and select the “View Event” selection button 162 in order to view more detailed information about the significant event or event matches. The conditions for qualifying events as significant events or event matches may be defined by the clinician. A significant event may be, for example, an arrhythmia faster than a certain threshold. A significant event matching may be, for example, an arrhythmia faster than a certain threshold that occurred within a certain amount of time of an EEG event. The significant events are typically represented by individual objects of graphical display, while the significant event “matches” are displayed as regions of overlap between the objects. The display on programmer 18 may be a touch screen, in which case the clinician may select selection button 162 by touching the representation of selection button 162 with a finger, a stylet or another device. Alternatively, the clinician may use a mouse, another peripheral device or another user interface to select a significant event with listing 160 and/or selection button 162.

The summary of the patient data for each event in the listing of events 164 may include an average EEG event per day or per month for the current session and a prior session, as well as the total number of each event. In the embodiment shown in FIG. 8, summary page 144 provides the total number of EEG events, patient marker events, and EKG events. The listing of therapy 165 provided by IMD 10 includes the total number of electrical stimulations provided to patient 12 by IMD 10 during a specified period of time (e.g., since the last session between programmer 18 and IMD 10 or for the overall life of IMD 10), as well as the average number of stimulations provided by IMD 10 per day during that specified period of time. In embodiments in which IMD 10 is a fluid delivery device, such as a drug pump, in addition to or instead of an electrical stimulator, therapy listing 165 of summary page 144 also provides the current prescription of drugs provided to the patient by IMD 10.

Diagnostic summary 166 of IMD 10 may provide any information about the operation of IMD 10 that the clinician may find useful. For example, in the embodiment shown in FIG. 8, diagnostic summary 166 provides the Elective Replacement Indicator (ERI) value, which is the estimated remaining life of the power source of IMD 10 if IMD 10 has a non-rechargeable power source. Diagnostic summary 166 may also provide the measured impedance of electrodes carried by leads 16 (FIG. 1). IMD 10 may be configured to measure the electrode impedance. Electrode impedance may be a useful diagnostic value of IMD 10 because electrical stimulation systems typically depend upon the ability of IMD 10, via electrodes of leads 16, to be able to convey electrical pulses of known energy to the target stimulation site. If the electrode impedance is too high or too low, the level of stimulation provided by the electrodes of leads 16 may be unsuitable for providing electrical stimulation therapy to patient 12.

Programmer 18 may automatically generate a dynamic graphical display 168 for inclusion in GUI 140. Graphical display 168 organizes the patient data into a Venn diagram, which displays the relationship between each of the events listed in the event listing 164. Graphical display 168 may help the clinician quickly sort through patient data retrieved from IMD 10 and visually ascertain relationships between different types of data.

As described above with reference to graphical displays 44, 70, and 90 of FIGS. 4-6, respectively, graphical display 168 also provides dynamic links to patient data within each of the sections of the Venn diagram. Upon receiving patient data from IMD 10 and reviewing summary page 144, the clinician may be interested in reviewing patient data for a specific combination of events. For example, if the clinician is interested in viewing events during a particular time period in which both a patient marker was generated and an EEG event occurred, where the “EEG event” may be a situation in which IMD 10 recorded an EEG value that was above a certain threshold, the clinician may merely select the relevant section of graphical display 168 to review the patient data corresponding to such patient data. In contrast, in many systems in which the patient data is presented in a linear format, the clinician may have to scroll through patient data and manually sort the data.

Patient data organized in graphical display 168 helps eliminate human error because identification of relevant data does not depend on the ability of the clinician to find the relevant data, but rather, programmer 18 automatically groups patient data into useful groups (i.e., the selectable sections of graphical display 168). Graphical display 168 also helps minimize the amount of time required to identify relevant information as compared to linear data formats.

Display pages 148 and 150 may include information relating to the stimulation settings for IMD and the location of leads 16 within patient 12. Display page 152 includes patient information, such a medical history, height, weight, contact information, and so forth. Finally, display page 154 summarizes changes that were made to the patient's therapy program during the current session. Display pages 144-154 are merely one embodiment of GUI 140, and in other embodiments, GUI 140 may include greater or fewer display pages 144-154. For example, in other embodiments, display pages 148 and 150, which display information relating to the therapy program for patient 12, may be combined into a single display page or may be implemented by a separate software program executed by programmer 18.

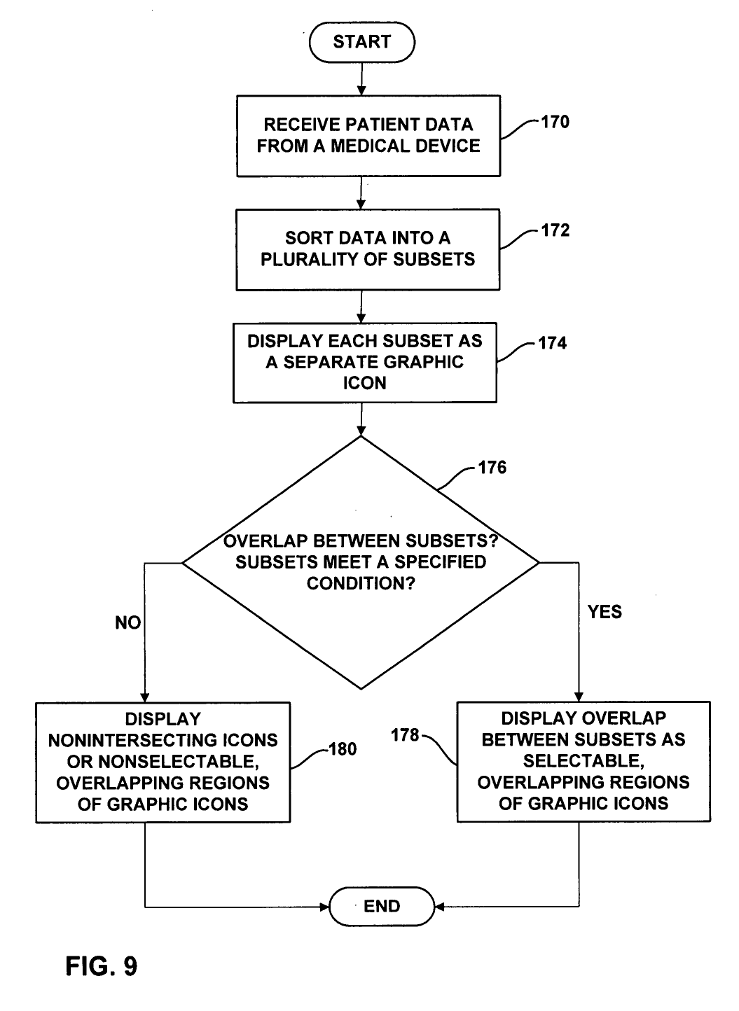

FIG. 9 is a flow diagram of one embodiment of a method for organizing patient data and displaying the patient data as a dynamic graphic display 44 (FIG. 5). While graphic display 44 is referred to throughout the description of FIG. 9, it should be understood that the method shown in FIG. 9 is also applicable to other types of dynamic graphic displays that organizes patient data into a Venn diagram format with selectable sections.

Patient data 32 (FIG. 3) is received from IMD 10 and stored in memory 31 (FIG. 3) of programmer 18 (FIG. 3) (170). Processor 30 (FIG. 3) may sort the received patient data 32 into different subsets of patient data (172). Processor 30 may utilize event condition definitions 33 stored in memory 32 of programmer 18 to derive events from physiological parameters, although processor 30 does not always derive events from physiological parameter measurements because a patient marker may also indicate an event. The patient data may be sorted into different subsets based on events, where one subset of patient data may be EEG parameter values that are above or below a respective threshold value, a second subset of patient data may be ECG parameter values that are above or below a respective threshold value, and a third subset of patient data may be the time and date of patient markers. Other events conditions may also be used. For example, as described in detail above, event conditions may be defined by certain characteristics of a waveform of the signal received from a physiological parameter sensor. The characteristic may include, for example, the slope, amplitude, temporal correlation or frequency correlation with a template signal.

Processor may generate graphic display 44 including a selectable graphic object for each subset of patient data (174). Simultaneously or thereafter, processor 30 may determine whether there are any overlaps in the subsets of patient data (176). “Overlaps” may be, for example, an overlap in time (i.e., did a data record from one subset of patient data and a data record from another subset occur at substantially the same time or within a certain time range of each other?). If there is an overlap between two subsets of patient data, processor 30 may generate graphic display 44 such that the relevant graphic objects 46, 48 or 50 intersect (178) to define one or more sections 52-64, where sections 58-64 are visually represented as overlapping regions between graphic objects 46, 48, and 50. As described above, a clinician may select a section 52-56 to obtain further details about the patient data within the respective subsets of data, and sections 58-64 between graphic objects 46, 48, and 50 are also selectable to obtain further details about the patient data that overlaps between the respective events.

If there is no overlap between data records of the subsets of data, processor 30 may generate graphic display 44 in which the relevant graphic objects do not intersect (180). For example, if events A and B do not overlap, graphic objects 46 and 48 of display 44 may not intersect, and display 44 may not include sections 58 and 64. Alternatively, if there is no overlap between the subsets of data, processor 30 may generate graphic display 44 in which the graphic objects 46, 48, and 50 overlap, but the sections 58-64 for which there is no overlap between the events are not selectable. As yet another alternative, the relevant overlapping sections 58-64 between the graphic objects may be selectable, but no information may be called up when the clinician or another user selects a section 58-64 associated with the non-overlapping events. For example, if events A and B do not overlap, graphical display 44 may include sections 58 and 64, but upon selection of sections 58, 64, a blank display may be shown because there is no patient data to display.

Various embodiments of the invention have been described. However, one of ordinary skill in the art will understand that various modifications may be made to the described embodiments without departing from the scope of the invention. For example, although the embodiments above primarily refer to a processor of a computing device, such as a patient programmer or a clinician programmer, deriving events from physiological parameter measurements, the invention is not so limited. In other embodiments, an implantable medical device may determine events from physiological parameter measurements and transmit the events instead of or in addition to the physiological parameter measurements to another computing device. In addition, although embodiments in which a graphical display includes selectable sections are described above, in other embodiments, the graphical display may not include selectable sections. Rather, graphical display may only display the sections to a clinician, such as to present an overview of the patient data tot he clinician. These and other embodiments are within the scope of the following claims.

1. A method comprising:
receiving patient data from a medical device;
generating a graphical user interface comprising a plurality of graphic objects defining at least:
a first section corresponding to a first subset of patient data;
a second section corresponding to a second subset of patient data; and
a third section corresponding to a conjunction of the first and second subsets of patient data; and
presenting the graphical user interface via a display.

2. The method of claim 1, wherein at least one of the first, second or third sections are selectable by a user.

3. The method of claim 1, further comprising:
receiving user input selecting one of the first, second or third sections; and
presenting output data corresponding to a respective subset of patient data within a selected one of the first, second or third sections.

4. The method of claim 1, wherein receiving patient data from the medical device comprises receiving patient data from an implantable medical device via wireless telemetry.

5. The method of claim 1, wherein receiving patient data from a medical device comprises receiving physiological parameter measurements from the medical device, the method further comprising deriving events from the physiological parameter measurements for inclusion in at least one of the first or second subsets of patient data.

6. The method of claim 5, wherein the physiological parameter comprises at least one of: electrocardiogram measures, electroencephalogram measures, blood pressure measurements, electrical activity of a heart of a patient, minute ventilation, cardiovascular pressure measures, venous oxygen levels, blood pressure patient activity level or body temperature.

7. The method of claim 5, wherein deriving events from the physiological parameter measurements comprises comparing at least one of the physiological parameter measurements to a threshold value.

8. The method of claim 5, wherein the physiological parameter measurements comprise measurements of a first type of physiological parameter, wherein deriving events comprises comparing a trend in the measurement of the first type of physiological parameter to a template.

9. The method of claim 8, wherein the trend indicates a rate of change of the measurement of the first type of physiological parameter over a period of time.

10. The method of claim 1, wherein the physiological parameter measurements comprise measurements of a first type of physiological parameter and a second type of physiological parameter, wherein deriving events comprises analyzing a first frequency component of a waveform of the measurement of the first type of physiological parameter and comparing the first frequency component to a second frequency component of a waveform template.

11. The method of claim 1, further comprising receiving an indication from a patient and generating a patient marker for inclusion in at least one of the first or second subsets of patient data upon receiving the indication from the patient.

12. The method of claim 1, wherein generating the graphical user interface comprises:
determining whether a first data record within the first subset of patient data and a second data record within the second subset of patient data occurred within a predetermined range of time; and
associating the first and second data records within the third section if the first and second data records occurred within the predetermined range of time.

13. The method of claim 12, wherein the predetermined time range is less than one minute.

14. The method of claim 1, wherein generating the graphical user interface comprises:
determining whether a first data record within the first subset of patient data and a second data record within the second subset of patient data occurred in a predetermined order; and
associating the first and second data records in with the third section if the first and second data records occurred in the predetermined order.

15. The method of claim 1, wherein the plurality of graphic objects define a Venn diagram.

16. A system comprising:
a display;
a user interface;
a telemetry module configured to receive patient data from a medical device; and
a processor configured to:
generate a graphical user interface comprising a plurality of graphic objects defining at least:
a first section corresponding to a first subset of patient data;
a second section corresponding to a second subset of patient data; and
a third section corresponding to a conjunction of the first and second subsets of patient data; and
display the graphical user interface on the display.

17. The system of claim 16, wherein at least one of the first, second or third sections is selectable by a user, and wherein the processor is further configured to:
receive user input selecting one of the first, second or third selectable sections; and
display a respective subset of patient data corresponding to the selected one of the first, second or third selectable sections.

18. The system of claim 16, wherein the processor is further configured to derive events from raw physiological parameter measurements.

19. The system of claim 18, wherein the processor derives the events by comparing at least one of the physiological parameter measurements to a threshold value.

20. The system of claim 18, wherein the physiological parameter measurements comprise measurements of a first type of physiological parameter and a second type of physiological parameter, wherein the processor derives the events by comparing a trend in the measurement of the first type of physiological parameter to a template.

21. The system of claim 18, wherein the physiological parameter measurements comprise measurements of a first type of physiological parameter and a second type of physiological parameter, wherein the processor derives the events by analyzing a first frequency component of a waveform of the measurement of the first type of physiological parameter and comparing the first frequency component to a second frequency component of a waveform template.

22. The system of claim 16, wherein at least one of the first or second subsets of patient data comprises patient markers.

23. The system of claim 16, wherein the conjunction of the first and second subsets of patient data comprises data records within the first and second subsets that occurred within a predetermined range of time as each other.

24. A graphical display comprising:
a first graphic object corresponding to a first subset of patient data;
a second graphic object corresponding to a second subset of patient data;
a third graphic object corresponding to a third subset of patient data; and
a fourth graphic object corresponding to a fourth subset of patient data,
wherein the first, second, third, and fourth graphical objects partially overlap,
wherein a first region of overlap between the first and third graphic objects corresponds to a conjunction of the first and third subsets of data, and
wherein the first region is represented as a subsection of the first graphical object, and the third graphic object defines a second region corresponding to the first region.

25. The graphical display of claim 24, wherein the second region comprises a void in the third graphic object.

26. A computer-readable medium comprising instructions to cause a processor to:
organize patient data received from a medical device into at least a first subset of patient data and second subset of patient data; and
generate a graphical user interface comprising plurality of graphic objects defining at least:
a first section corresponding to the first subset of patient data;
a second section corresponding to the second subset of patient data; and
a third section corresponding to a conjunction of the first and second subsets of patient data.

27. The computer-readable medium of claim 26, further comprising instructions to cause the processor to:
determine whether a first data record within the first subset of patient data and a second data record within the second subset of patient data occurred within a predetermined range of time; and
associate the first and second data records in with the third section if the first and second data records occurred within the predetermined range of time.

28. The computer-readable medium of claim 26, further comprising instructions to cause the processor to:
receive user input selecting one of the first, second or third sections; and
present information corresponding to a respective subset of patient data within a selected one of the first, second or third sections.
